(19) United States
(12) Patent Application Publication
(10) Pub. No.: US 2005/0281755 A1
(43) Pub. Date: Dec. 22, 2005

(54) Topical foam/mousse compositions for treating psoriasis

(75) Inventors: Leila Zarif, Valbonne (FR); Claire Mallard, Mougins (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GALDERMA S.A., CHAM (CH)

(21) Appl. No.: 10/965,195

(22) Filed: Oct. 15, 2004

(30) Foreign Application Priority Data
Jun. 17, 2004 (FR) 04/06613

(51) Int. Cl. A61L 9/04; A61K 9/00; A61K 31/59

(52) U.S. Cl. 424/47; 514/167; 514/171

(57) ABSTRACT

Topically applicable, pharmaceutical foam/mousse compositions, well suited for the treatment of psoriasis, include a hydrophobic phase, at least one surfactant, a therapeutically effective amount of a vitamin D analogue and a therapeutically effective amount of a corticosteroid.

### CROSS-REFERENCE TO PRIORITY APPLICATION

This application claims priority under 35 U.S.C. § 119 of FR 04/06613, filed Jun. 17, 2004, hereby expressly incorporated by reference and assigned to the assignee hereof.

### BACKGROUND OF THE INVENTION

1. Technical Field of the Invention

The present invention relates to topical compositions for treating psoriasis, and which comprise a corticosteroid and a vitamin D analogue.

2. Description of Background and/or Related and/or Prior Art

Psoriasis is a chronic inflammatory skin disease that affects about 5% of the French population. This disease is manifested by red plaques covered with whitish flakes which detach from the skin: these are squamae. Psoriasis plaques are often localized at the elbows, the scalp and the knees, but may also affect other parts of the body, for instance the face, the hands, the feet and mucous membranes. Psoriasis is neither contagious nor of allergic nature, but it is liable to be transmitted by heredity, in the form of a susceptibility towards developing the disease. Psoriasis may occur at any age, but the first outbreaks mainly occur between the ages of 10 and 30. It is a chronic disease whose development is unpredictable: relapse phases are followed by remission phases. Although this disease rarely places an individual's life in danger, it does, however, have a high impact on the quality of life. With regard to its unaesthetic appearance and its chronic nature, the disease often gives rise to feelings of self-deprecation, injury to the morale and, gradually, depression. Individuals suffering from psoriasis often have difficulties in communication, most particularly when their lesions are visible to others: this is especially the case for psoriasis of the face, the scalp and the hands.

Psychological trauma (grieving, emotional breakup, etc.) and physical shocks (accidents, surgery, etc.) are often the cause of the first outbreaks and of relapses.

Two types of psoriasis are distinguished:
type I, in which the disease develops in children and young adults, with a family history and a quite severe evolution,
type II, in which the psoriasis develops after the age of 40, without a family history and with a more benign evolution.

In psoriasis, certain individuals suffer from a single psoriasis plaque located in a specific region of the body, while others are subject to psoriasis spread throughout the body. Similarly, there are several types of lesion, giving rise to quite distinct forms of psoriasis.

In the prior art, it is common practice to use corticosteroids to treat psoriasis. The mechanism of action of corticosteroids is attributed to their inhibition of inflammatory processes (Lange K. et al., Skin Pharmacol. Appl. Skin Physiol., 13(2): 93-103 (2000)).

U.S. Pat. No. 4,610,978 describes the use of vitamin D or a vitamin D analogue, optionally combined with a corticosteroid, for treating psoriasis. It is known practice at the present time to use a combination of active agents in the treatment of psoriasis, and especially a combination of a corticosteroid and vitamin D or a vitamin D analogue. Specifically, the combined therapy is advantageous since it enables the doses of active agents administered to be reduced, and thus allows a reduction in the side effects of these active agents.

WO 00/64450 describes, for the treatment of psoriasis, a pharmaceutical composition for dermal application comprising at least one vitamin D analogue and at least one corticosteroid. These compositions are presented in the form of lotions or creams.

WO 02/34235 describes, for the treatment of psoriasis, a pharmaceutical composition in gel form for application to the skin, comprising at least one vitamin D analogue, at least one corticosteroid and a viscosity-increasing excipient.

However, the prior art to date has not described or suggested a composition for treating psoriasis that is in the form of a mousse and that contains a combination of a vitamin D analogue and a corticosteroid.

One skilled in the art would not have considered combining agents of vitamin D analogue type with a corticosteroid in a stable foaming form.

### SUMMARY OF THE INVENTION

The present invention thus features stable foaming pharmaceutical compositions for topical application, for treating psoriasis, comprising at least one hydrophobic phase, a surfactant and, as active principle, a combination of a vitamin D analogue such as calcitriol and of a corticosteroid such as clobetasol propionate. The foaming pharmaceutical composition may optionally comprise a co-surfactant and an organic solvent.

The foaming pharmaceutical compositions of the invention present numerous advantages. Specifically, given that mousses are easy to apply, especially to the scalp, they make it possible to improve the patient's compliance with the treatment.

In the present patent application, the expression “composition for topical application” means a composition intended to be applied to any part of the body, such as the scalp, mucous membranes, the elbows, the knees, the hands, the feet, the face, etc.

### DETAILED DESCRIPTION OF BEST MODE AND SPECIFIC/PREFERRED EMBODIMENTS OF THE INVENTION

The hydrophobic phase, also referred to hereinbelow as the hydrophobic solvent, may be, without this being limiting however, a plant oil, a mineral oil that is liquid or solid at room temperature, or a silicone oil. The hydrophobic phase may act as solvent for the active agents(s).

Moreover, the active agent may be dissolved in an organic solvent that is different from the hydrophobic phase. This solvent may be a glycol derivative, for instance propylene glycol, a fatty acid ester, for instance an alkyl benzoate containing a C12-C15 alkyl chain, a medium- or long-chain alcohol, an aromatic or alkylated pyrrolidinone, a cyclic ketone, a cyclic ether or an alkane containing a linear, branched or cyclic chain.

Examples of plant oils that may be mentioned include soybean oil and cottonseed oil. Mineral oils that may be mentioned include lanolin oil, isopropyl palmitate, octyl palmitate, isostearic acid derivatives, neopentyl glycol dicaprylate/dicaprate and hydrogenated glycerides. Examples of silicone oils that may be mentioned include non-volatile silicones, for instance polyalkyl siloxanes and polyaryl siloxanes.

The hydrophobic solvent is advantageously present in a concentration ranging from 20% to 75% (w/w).

Examples of vitamin D analogues that may be mentioned include calcitriol, tacalcitol, calcipotriol and any other vitamin D analogue mentioned in WO 00/64450. The vitamin D analogue is preferably calcitriol.

Examples of corticosteroids that may be mentioned include clobetasol and its esters such as clobetasol 17-propionate (also referred to hereinbelow as clobetasol propionate), betamethasone and its esters, fluocinonide, hydrocortisone and any other corticosteroid mentioned in WO 00/64450. The corticosteroid is preferably clobetasol propionate.

The surfactant may be a nonionic, zwitterionic, anionic or cationic surfactant, or a mixture of these surfactants.

These surfactants are known to those skilled in the art, for instance sorbitan esters, sucrose esters, pegylated esters, sodium lauryl sulfate and betaines.

The surfactant is preferably nonionic.

The co-surfactant is selected from co-surfactants with an HLB of from 6 to 10 and preferably from 6 to 8.

The present invention also features compositions comprising at least one propellent gas. This propellent gas is selected from the group consisting of propane, butane, dichlorodifluoromethane, dichlorotetrafluoroethane and octafluorocyclobutane, or mixtures thereof.

According to one preferred embodiment of the invention, the propellent gas is in liquefied form/state and its concentration is from 5-30% of the total composition.

The compositions that are the subject of the present invention may also comprise a wetting agent, a suitable buffer substance and/or an antioxidant.

Examples of wetting agents that may be mentioned include glycerol, panthenol and sorbitol.

Examples of buffer substances that may be mentioned include acetic acid/sodium acetate, citric acid/sodium citrate, phosphoric acid/sodium phosphate or anhydrous citric acid/potassium citrate couples.

The antioxidant may be a 4-aminosalicylic acid, a 5-aminosalicylic acid, butyl hydroxy toluene, butyl hydroxy anisole or an α-tocopherol and derivatives thereof.

According to one preferred embodiment of the invention, the surfactant is present in an amount ranging from 0.1% to 15% by weight relative to the total weight of the composition.

The vitamin D analogue is advantageously present in an amount of from 0.0001% to 1%, preferably from 0.0001% to 0.1% and most preferably from 0.0001% to 0.025% by weight relative to the total weight of the composition.

The corticosteroid is advantageously present in an amount ranging from 0.001% to 1%, preferably from 0.001% to 0.2% and most preferably from 0.005% to 0.1% by weight relative to the total weight of the composition.

The propellent gas is advantageously present in an amount ranging from 5% to 30% and preferably from 5% to 10% by weight relative to the total weight of the composition.

The hydrophobic phase is advantageously present in an amount ranging from 20% to 75% and preferably from 20% to 40% by weight relative to the total weight of the composition.

The present invention also features an aerosol can or dispenser comprising a composition as defined above.

The present invention also features a process for preparing a foaming pharmaceutical composition as defined above, in an aerosol can.

The process for preparing the foaming compositions that are the subject of the present invention comprises the following steps:
(a) the active agents are separately dissolved in a suitable solvent;
(b) the hydrophobic phase is heated, if necessary, to 50-70° C.;
(c) the dissolved active agents are added to the hydrophobic phase with stirring;
(d) the aqueous phase containing the surfactant preheated to the same temperature (if necessary) is added gently to the hydrophobic phase;
(e) the mixture is homogenized with an Ultra-Turrax blender and cooled to room temperature;
(f) the mixture is then placed in an aerosol, the aerosol container is sealed and the required amount of propellant (about 10% of the composition by mass) is compressed into the container.

The present invention also features a mixture of a vitamin D analogue and of a corticosteroid for the manufacture of a foaming pharmaceutical composition for topical application, in a regime or regimen for treating psoriasis. 5

In order to further illustrate the present invention and the advantages thereof, the following specific example is given, it being understood that same is intended only as illustrative and in nowise limitative. In said example to follow, all parts and percentages are given by weight, unless otherwise indicated.

### EXAMPLE 1

| Ingredient | Percentage |
|---|---|
| Miglyol 812 | 50% |
| Water | 30% |
| Propylene glycol | 5% |
| Sucrose ester | 3% |
| Antioxidant | 0.02% |
| Preservatives | 0.5% |
| Calcitriol | 0.0003% |
| Clobetasol propionate | 0.02% |
| Propellant | 10% |

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A foaming pharmaceutical composition suited for the treatment of psoriasis, comprising a hydrophobic phase, at least one surfactant, a therapeutically effective amount of a vitamin D analogue and a therapeutically effective amount of a corticosteroid.

2. The foaming pharmaceutical composition as defined by claim 1, comprising a propellant gas therefor.

3. The foaming pharmaceutical composition as defined by claim 2, said propellant gas being in liquefied state.

4. A stable foam/mousse pharmaceutical composition suited for the treatment of psoriasis, comprising a hydrophobic phase, at least one surfactant, a therapeutically effective amount of a vitamin D analogue and a therapeutically effective amount of a corticosteroid.

5. The pharmaceutical composition as defined by claims 1 or 4, further comprising a co-surfactant.

6. The pharmaceutical composition as defined by claims 1 or 4, further comprising an organic solvent.

7. The pharmaceutical composition as defined by claims 1 or 4, said vitamin D analogue comprising calcitriol, calcipotriol or tacalcitol.

8. The pharmaceutical composition as defined by claim 7, said vitamin D analogue comprising calcitriol.

9. The pharmaceutical composition as defined by claim 7, said corticosteroid comprising clobetasol propionate, betamethasone and its esters, fluocinonide or hydrocortisone.

10. The pharmaceutical composition as defined by claim 9, said corticosteroid comprising clobetasol propionate.

11. The pharmaceutical composition as defined by claims 1 or 4, said at least one surfactant comprising a nonionic, anionic, zwitterionic or cationic surfactant.

12. The pharmaceutical composition as defined by claim 11, comprising a nonionic surfactant selected from the group consisting of ethoxylated sorbitan stearate, ethoxylated sorbitan palmitate, ethoxylated sorbitan oleate, ethoxylated nonylphenol, ethoxylated fatty alcohols, polyoxyethylene lauryl ether, polyoxyethylene cetyl ether and sucrose esters, or mixtures thereof.

13. The pharmaceutical composition as defined by claim 11, comprising a zwitterionic surfactant selected from the group consisting of a cocamidoalkylamine, a cocamidopropylamine and a cocamidopropylamine oxide.

14. The pharmaceutical composition as defined by claim 11, comprising a betaine cationic surfactant.

15. The pharmaceutical composition as defined by claim 2, said propellant gas being selected from the group consisting of propane, butane, dichlorodifluoromethane, dichlorotetrafluoroethane and octafluorocyclobutane, or mixtures thereof.

16. The pharmaceutical composition as defined by claims 1 or 4, comprising at least one hydrophobic solvent.

17. The pharmaceutical composition as defined by claim 16, said hydrophobic solvent being selected from the group consisting of a mineral oil, a silicone oil and a plant oil, or mixtures thereof.

18. The pharmaceutical composition as defined by claims 1 or 4, said vitamin D analogue comprising from 0.0001% to 1% by weight thereof.

19. The pharmaceutical composition as defined by claims 1 or 4, said vitamin D analogue comprising from 0.0001% to 0.1% by weight thereof.

20. The pharmaceutical composition as defined by claims 1 or 4, said vitamin D analogue comprising from 0.0001% to 0.025% by weight thereof.

21. The pharmaceutical composition as defined by claim 18, said corticosteroid comprising from 0.001% to 1% by weight thereof.

22. The pharmaceutical composition as defined by claim 18, said corticosteroid comprising from 0.001% to 0.2% by weight thereof.

23. The pharmaceutical composition as defined by claim 18, said corticosteroid comprising from 0.005% to 0.1% by weight thereof.

24. The pharmaceutical composition as defined by claim 11, said at least one surfactant comprising from 0.1% to 15% by weight thereof.

25. The pharmaceutical composition as defined by claim 11, said at least one surfactant comprising from 0.2% to 5% by weight thereof.

26. The pharmaceutical composition as defined by claim 2, said propellant gas comprising from 3% to 30% by weight thereof.

27. The pharmaceutical composition as defined by claim 2, said propellant gas comprising from 3% to 10% by weight thereof.

28. The pharmaceutical composition as defined by claim 2, said propellant gas comprising from 3% to 5% by weight thereof.

29. The pharmaceutical composition as defined by claim 16, said at least one hydrophobic solvent comprising from 20% to 75% by weight thereof.

30. The pharmaceutical composition as defined by claim 16, said at least one hydrophobic solvent comprising from 20% to 40% by weight thereof.

31. The pharmaceutical composition as defined by claims 1 or 4, further comprising a wetting agent, a buffer and/or an antioxidant.

32. A regime or regimen for the treatment of psoriasis comprising topically applying onto the affected skin area of an individual afflicted with psoriasis, a thus effective amount of the foam/mousse composition as defined by claim 4.

33. An aerosol dispenser comprising a housing confining a foaming pharmaceutical composition as defined by claim 1 and a gaseous propellant for spraying a foam out of said housing.
